(19) United States
(12) Patent Application Publication
(10) Pub. No.: US 2006/0140906 A1
(43) Pub. Date: Jun. 29, 2006

(54) Composition comprising cysteamine for improving immunity of animals

(76) Inventors: Francis Chi, Hong Kong (CN); Qin Tang Wen, Hong Kong (CN)

Correspondence Address: SMITH-HILL AND BEDELL, P.C., 16100 NW CORNELL ROAD, SUITE 220, BEAVERTON, OR, 97006, US

(21) Appl. No.: 10/546,127

(22) PCT Filed: Feb. 17, 2004
(86) PCT No.: PCT/EP04/01476

§ 371 Date: Aug. 19, 2005

(30) Foreign Application Priority Data
Feb. 19, 2003 (EP) 0303843.7

(51) Int. Cl. A61K 38/20; A61K 31/13; A61K 31/724

(52) U.S. Cl. 424/85.2; 514/665; 514/58

(57) ABSTRACT

There is provided a new use of cysteamine, a salt thereof, or a composition containing cysteamine or a salt thereof, for improving immunity of animals.

The present invention relates to the use of cysteamine or a cysteamine-containing composition for modulating immunity of animals, and in particular, improving immunity of vertebrate animals.

The survival of animals relies on an effective immune system. The immune system of animals and in particular vertebrate animals, made up of many organs and cells, defends the body against infection, disease and foreign substances such as viruses and bacteria. There have been proposed various compositions for boosting the immunity of the body for treating diseases or simply to improve the general well being of the body. For example, some pharmaceuticals for HIV infection treat the HIV-infected patients by seeking to improve the general immunity of the patients. However, these pharmaceuticals are not always reliable and they tend to cause many undesirable side effects.

It is thus an object of the present invention in which the above issues are addressed, or at least to provide a useful alternative to the public. The present invention provides a composition for use in modulating the immunity of animals; the composition may be used by itself or in combination with other existing compositions or pharmaceuticals.

According to a first aspect of the present invention, there is provided the use of cysteamine, a salt thereof, or a composition containing cysteamine or a salt thereof, for improving immunity of animals.

According to a second aspect of the present invention, there is provided a composition for improving immunity of animals, comprising cysteamine, a salt thereof, or a composition containing cysteamine or a salt thereof.

According to a third aspect of the present invention, there is provided an animal feed additive for improving immunity of animals comprising a composition as described above.

According to a fourth aspect of the present invention, there is provided an animal feed for improving immunity of animals comprising a composition as described above.

According to a fifth aspect of the present invention, there is provided a method of improving immunity of animals, comprising administering cysteamine, or a salt thereof, or a composition containing cysteamine or a salt thereof, into the animals.

Improving immunity may include increasing the level of interleukin-2 (IL-2) in the animals. Improving immunity may also include increasing the level of interleukin-6 (IL-6) in the animals. In particular, improving immunity may also include stimulating production of lymphocytes in the animals. Studies have shown that a higher level of IL-2 or IL-6 corresponds to a generally stronger immune system. It has been identified that the above use is particularly effective in vertebrate animals such as cattle and poultry.

Preferably, the composition may comprise 1 to 95 wt % of cysteamine, or a salt thereof, and an inclusion compound host materials composition comprising a stabilizer selected from a group including cyclodextrin and its derivatives. Suitably, the cysteamine-containing composition may comprise 1 to 75 wt % of cysteamine or a salt thereof. More suitably, the cysteamine-containing composition may comprise 1 to 40 wt % cysteamine or a salt thereof.

Preferably, the cysteamine-containing composition may comprise 1 to 60 wt % of the inclusion compound host materials composition. Suitably, the cysteamine-containing composition may comprise 10 to 40 wt % of the inclusion compound host materials composition. The stabilizer comprised in the inclusion compound host materials composition may be selected from a group including cyclodextrin (-CD), methyl β-cyclodextrin (M-β-CD), hydropropyl β-cyclodextrin (HP-β-CD), hydroethyl β-cyclodextrin (HE-β-CD), poly-cyclodextrin, ethyl β-cyclodextrin (E-β-CD) and branched cyclodextrin.

Preferably, the cysteamine-containing composition may comprise at least one of fillers, disintegrants, binders, flavorings and smelling agents, and coating materials. In particular, the cysteamine-containing composition may comprise 1 to 20 wt % of the coating materials. Advantageously, the coating materials may be enteric, and selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymer and formogelatine.

Preferably, the cysteamine-containing composition may comprise 1 to 90 wt % of the fillers. Suitably, the cysteamine-containing composition may comprise 1 to 60 wt % of the fillers, and the fillers may be selected from a group including powdered cellulose, starch and calcium sulfate.

Preferably, the cysteamine-containing composition may comprise 5 to 50 wt % of the binders and the disintegrants. Suitably, the cysteamine-containing composition may comprise 15 to 35 wt % of the binders and the disintegrants. The binders and the disintegrants may be selected from a group including hydropropyl starch, microbial alginate, microcrystalline cellulose and starch.

Preferably, the cysteamine-containing composition may comprise 0.05 to 0.3 wt % of the flavoring and smelling agents for enhancing the flavor thereof.

Suitably, the cysteamine-containing composition may be formed into granules, each of which comprises at least one layer of the coating materials. The cysteamine-containing composition may preferably be formed into granules in which the cysteamine or a salt thereof is shielded from its surroundings by the inclusion compound host materials composition. Each of the granules of the cysteamine-containing composition may have a size ranging from 0.28 to 0.90 mm in diameter. The granules with this range of size can be more easily swallowed by the animals. The cysteamine-containing composition may preferably be encapsulated by the enteric coating materials.

The cysteamine-containing composition may be used for the manufacture of an animal feed additive. The cysteamine-containing composition may also be used for the manufacture of an animal feed. In particular, the cysteamine-containing composition may be adapted for oral administration into animals. However, cysteamine for improving immunity in animals may be administered to animals by other means such as by direct injection.

The present invention is based on the demonstration that cysteamine or a cysteamine-containing composition when administered to animals such as cows has activity in modulating immunity and in particular improving immunity thereof. Prior to this finding, there was no suggestion or sufficient indication that cysteamine or its variants or derivatives might have such activity. The present invention also provides a feed and a feed additive for improving immunity of animals and a method for improving immunity of animals.

One effect of cysteamine is described in PRC Patent Publication No. CN 1358499 and International Patent Publication No. WO02/48110, the content of which is incorporated herein. However, a previously unknown effect of cysteamine is its effect on improving immunity of animals. The effect of cysteamine or a cysteamine-containing composition on the immunity of animals is explained as follows. It is believed that cysteamine having a physiological activity acts as a growth stimulator. Natural cysteamine is a part of coenzyme A (also know as CoA-SH or CoA) which is a coenzyme pattern of pantothenic acid. In the course of metabolism, coenzyme A acts as the carrier of dihydrosulfuryl or variants of hydrosulfuryl which is linked with the hydrosulfuryl of coenzyme A.

Experiments performed on other animals such as pigs, poultry, fowls, goats, rabbits and fish have shown that cysteamine can deplete somatostain (SS) in the animals. This increases the level of growth hormone in the blood of the animals which at the same time raises the level of various other growth stimulating factors including insulin-like growth factor I (IGF-I), insulin, triiodothyronine (T3), trthyroxine (T4) and beta-endorphin (beta-END).

As regards the present invention, it is shown that cysteamine can improve immunity by increasing the level of interleukin-2 (IL-2) and/or interleukin-6 (IL-6).

IL-2 belongs to a class of soluble, regulatory proteins known as cytokines. IL-2 is a 133-amino acid glycoprotein secreted by T(H) lymphocytes and other cells following activation by antigens, mitogens and other cytokines. IL-2 stimulates the proliferation and cytotoxicity of T lymphocytes, enhances the microbicidal and cytotoxic activities of NK cells, B-lymphocytes, macrophages and monocytes (Sule N S, Nerurkar R P, Kamath S; 2001; Interleukin-2 as a therapeutic agent; J Assoc Physicians India. 49: 897-900). It is shown that IL-2 represents an important role in the immune system.

Studies have shown that IL-2 leads to the curtailment of HIV spreading in HIV infected patients of all stages of HIV disease (Paredes R, Lopez Benaldo de Quiros J C, Fernandez-Cruz E, Clotet B, Lane H C; 2002; The potential role of interleukin-2 in patients with HIV infection. AIDS Rev 4(1): 36-40). IL-2 is also a promising immunotherapeutic agent mediating the regression of established growing cancers (Rosenberg S A. 200; Progress in the development of immunotherapy for the treatment of patients with cancer; J Intern Med 250(6): 462-75), metastatic melanoma, acute myelogenous leukemia and metastatic renal cell carcinoma (Atkins M B; 2002; Interleukin-2: clinical applications; Semin Oncol 29(3 Suppl 7): 12-7 )in humans. However, at the time of filing this application, it has been about fifteen years since the first positive clinical reports of IL-2 appeared in the medical literature, ten years since moderate dose continuous infusion IL-2 was approved in Europe and five years since high-dose bolus IL-2 was approved for general use in the United States when IL-2 has been accepted as a standard treatment to be used alone or in combination with chemotherapy or biotherapy in the management of diseases (Dillman R O. 1999. What to do with IL-2? Cancer Biother Radiopharm 14(6): 423-34). It took a relatively long time before IL-2 has been widely accepted to be used for improving the immunity in animals. Numerous reports have shown the immunomodulatory capabilities of recombinatant (rb) IL-2 on important mammary immune cell functions. In vitro and in vivo studies indicate that rbIL-2 markedly enhances the proliferation of mononuclear cells (Torre, P M. Konur P K. and Oliver S P; 1992; Proliferative response of mammary gland mononuclear cells to recombinatant bovine interleukin-2; Vet; Immunol. Immunopathol. 32: 351). Studies have shown that lymphocyte populations isolated from mammary tissues increase cytotoxic and bactericidal activities following in vitro culture with IL-2 (Shafer-Weaver K A. and Sordillo L M; 1996; Enhancing bactericidal activity of bovine lymphoid cells during the peripartument period; J. Dairy Sci. 79: 1347)(Sordillo L M. Campos M. and Babiuk L A; 1991; Antibacterial activity of bovine mammary gland lymphocytes following treatment with interluekine-2; J. Dairy Sci. 74: 3370).

Turning to IL-6, IL-6 has effects on most of the major effector cells of the immune system, including B-lymphocytes, helper T-lymphocytes, cytotoxic T-lymphocytes, and killer cells. In B-lymphocytes cultured in vitro, IL-6 is essential as a late-stage differentiation factor during the transition of B-cells into immunoglobulin-secreting plasma cells. Furthermore, IL-6 has been shown to augment secondary, but not primary, antibody responses in vivo. These and other data suggested that IL-6 plays a major role in the development of antigen-specific immune responses.

Many studies have shown that IL-6 can affect the proliferation of both peripheral and thymic T-cells. Furthermore, IL-6 can stimulate the differentiation of cytotoxic T-cells from the thymic precursors. IL-6 has been shown to augment the activity of natural killer cells.

Recent studies on cytokine function in hematopoiesis have shown that IL-6 cooperates with interleukin-3 (IL-3) in enhancing stem-cell differentiation.

The role of IL-6 in vivo in inflammation is to initiate the hepatic responses of production of acute-phase protein, such as collagenase, stromelysin, and the 72-kDa gelatinase, which enzymatically degrade connective tissue matrix. IL-6 probably also plays a role in inflammation at local tissue sites by modulating the balance of tissue-degrading activity. (Richards C D, Scamurra R W, and Murtaugh M P. Interleukin-6. p 155-157. In: Cytokines in animal health and disease. 1995. Edited by Michael J. Myers and Michael P. Murtaugh. Marcel Deckker, Inc. New York, Basel, Hong Kong)

Two kinds of effector mechanisms mediate immune responses. Specific molecules, called antibodies, mediate some immune responses. The synthesis of antibodies occurs in a subset of lymphocytes called B-lymphocytes or B-cells. Antibody-mediated immunity is called humoral immunity. Other immune responses are mediated by cells. All types of leukocytes in the blood participate in cell-mediated immunity. However, the specificity of the response depends upon a subset of lymphocytes called T-lymphocytes or T-cells. The mature T-cells include Helper T-cell, cytotoxic suppressor T-cell, and memory T-cell. Helper T-cells are required to initiate immune responses by recognizing foreign antigens and releasing factors that will promote the responses of other lymphocytes (B-cells and T-cells) to that antigen. Cytotoxic T-cells, on coming into contact with a target cell, deliver a “lethal hit” to destroy target cells, such as tumor or foreign cells.

Certain natural compounds have the ability to cause lymphocytes to divide. The most important of these compounds is a family of proteins called lectins, which are usually isolated from plants. Examples of lectins include phytohemagglutnin (PHA) which is obtainable from red kidney bean (Phaseolus vulgaris), and concanavalin A (Con A) which is obtainable from jack bean (canavalis ensiformis). These lectins have the ability to specifically bind sugars on lymphocyte membrane to stimulate nucleoside incorporation, phospholipid synthesis, DNA synthesis, and cell division. PHA stimulates primarily T-cell division. These mitogens can be used to assist in the differentiation of T-cells and, by measurement of the response provoked, demonstrate the ability of the T-cell system to respond to nonimmunologic stimuli (Sule N S, Nerurkar R P, Kamath S. 2001. Interleukin-2 as a therapeutic agent. J Assoc Physicians India. 49: 897-900).

The present invention teaches the use of cysteamine for improving immunity of animals. It is believed that cysteamine depletes the level of somatostatin and increases the level of growth hormone, and this results in an increase of mitogen-induced lymphocyte proliferation. In particular, the present invention uses a cysteamine-containing composition, a compound of cysteamine enveloped in microcapsule. The following experiments seek to demonstrate the effect of cysteamine or the cysteamine-containing composition on the immune system and immunity of animals. The experiments also seek to demonstrate the effect of cysteamine or the cysteamine-containing composition on the concentration of serum IL-2 and the concentration of plasma IL-6 in the animals.

The cysteamine-containing composition suitable for use in the present invention comprises two main ingredients of 1 to 95 wt % of cysteamine (or its salts, for example, cysteamine hydrochloride, or other pharmaceutically acceptable acid addition salts thereof) and 1 to 80 wt % of a carrier such as inclusion compound host materials. The chemical formula of cysteamine is HSCH₂CH₂NH₂. The term “cysteamine” referred hereinafter means cysteamine and/or its salt like compounds. Cysteamine and its salts are well known in the chemical literature.

The general chemical formula of a cysteamine salt is C₂H₇NS.X, where X may be HCl, H₃PO₄, bitartrate, salicylate, etc. The cysteamine used is preferably of pharmaceutically acceptable standard and the content of carbon, hydrogen, nitrogen and sulfur therein are substantially 31.14 wt %, 9.15 wt %, 18.16 wt % and 41.56 wt % respectively. While the workable content of cysteamine in the cysteamine-containing composition ranges from 1 to 95 wt %, a preferable range of 1 to 75 wt % and a more preferable range of 1 to 40 wt % of cysteamine may be used. Cysteamine is one of the main active ingredients of the cysteamine-containing composition. However, it has been identified that if the content of cysteamine in the cysteamine-containing composition exceeds 95 wt %, mixing the composition with a basal feed would be rather difficult and the effect of the composition for improving immunity of the animals would be hindered.

The inclusion compound host materials may comprise mainly cyclodextrin and/or its derivatives which are selected from a group including methyl β-cyclodextrin (M-β-CD), hydropropyl β-cyclodextrin (HP-β-CD), hydroethyl β-cyclodextrin (HE-β-CD), polycyclodextrin, ethyl β-cyclodextrin cyclodextrin (E-β-CD) and branched cyclodextrin. The general chemical formula of cyclodextrin is (C₆O₅H₉)ₙ.(C₆O₅H₉)₂ and the structural formula is as follows. 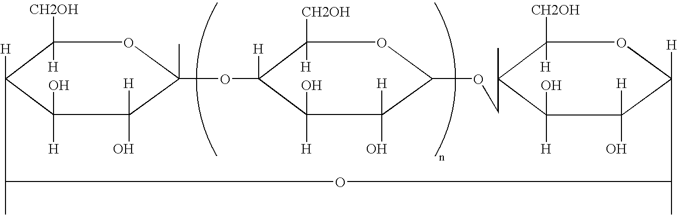
where α-CD n=4; β-CD n=5; γ-CD n=6. (Cyclodextrin is a cyclic oligomer of alpha-D-glucopyranose.)

It is worthwhile to note that the β-CD form of cyclodextrin is preferably used because the internal diameter of its molecule is about 6-8 Å which makes it a particular suitable candidate as an inclusion compound host material for preparation of the cysteamine-containing composition, which involves the use of an inclusion process. The term “cyclodextrin” referred hereinafter means cyclodextrin and/or its derivatives. Any derivative of cyclodextrin which has the property of stabilizing and protecting cysteamine from degradation may be used. For example, any one of the groups of cyclodextrin or its derivatives mentioned above may be used.

While the workable content of the inclusion compound host materials in the cysteamine-containing composition ranges from 1 to 80 wt %, a preferable workable range of 1 to 60 wt % and a more preferable workable range of 10 to 40 wt % of the inclusion compound host materials may also be used. The actual amount of the inclusion compound host materials used will depend on the actual content of the cysteamine used in preparing the cysteamine-containing composition.

The cysteamine-containing composition may also comprise 1 to 90 wt % of fillers although a preferable workable range of 1 to 60 wt % and a more preferable workable range of 1 to 40 wt % of the fillers may also be used in the composition. The actual content will depend on the actual amount of cysteamine and inclusion compound host materials used. The fillers may be selected from a group including powdered cellulose, starch and calcium sulfate (e.g. CaSO₄.2H₂O). It is to be noted that if the content of the fillers exceeds 90 wt % in the cysteamine-containing composition, the content of the main active ingredients will thus be reduced, and the cysteamine-containing composition may become less effective in improving immunity of the animals administered therewith.

The cysteamine-containing composition may also comprise 5 to 50 wt % of disintegrants and binders although a preferable workable range of 10 to 40 wt % and a more preferable workable range of 15 to 35 wt % may also be used. The actual content will depend on the actual amount of cysteamine, the inclusion compound host material and other ingredients used. The binders and disintegrants may be selected from a group including hydropropyl starch, microbial alginate, microcrystalline cellulose and starch. It has been identified that if the content of the disintegrants and binders in the composition is less than 5 wt %, granules of the composition produced will lack the required hardness.

In addition, manufacturing of the composition would become very difficult. If however the content of the disintegrants and binders is more than 50 wt %, the resulting composition will have excessive hardness, this is especially so if the content of binders represents a large portion of the mixture of the disintegrants and binders. This will result in difficult absorption of the composition by the intestines of the animals.

The cysteamine-containing composition may also comprise 0.05 to 0.3 wt % of flavoring and smelling agents which may be a flavoring essence.

The cysteamine-containing composition may also comprise 1 to 20 wt % of coating materials although a preferable workable range is 1 to 15 wt % and a more preferable workable range is 2 to 10 wt %. The actual content will depend on the actual amount of cysteamine, the inclusion compound host materials and the other ingredients used. The coating materials are preferably enteric-coated which allows dissolution in an alkaline environment such as in the intestines. The coating materials may be made of and selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymers, lac and formogelatine. It has been identified that if the content of the coating materials is less than 1 wt %, granules of the composition may not be entirely covered by the coating materials which act as a protective layer. The cysteamine-containing composition may thus degrade before being absorbed by the intestines into the bloodstream of the animals. On the other hand, if the content of the coating materials exceeds 15 wt %, the active ingredients in the composition may not effectively be released from the composition. Thus, the intended modulation of immunity would not be achieved.

The cysteamine-containing composition used in the present invention is in the form of small granules each of which has a preferable diameter of substantially 0.28 to 0.90 mm. These granules are prepared using a micro-encapsulation method. The method involves using a macromolecular substance having inclusion property. The macromolecular substance may be the inclusion compound host materials (which may comprise mainly cyclodextrin) as described above. The macromolecular substance acts as a molecular capsule to engulf the molecules of cysteamine, whereby cysteamine in the composition is protected and insulated from light, heat, air and moisture of the surroundings. The stability of cysteamine is thus preserved. The inclusion compound host materials used in the micro-encapsulation method preferably comprises a cyclic polysaccharide compound having 6 to 12 glucose molecules, which is produced by reacting cyclodextrin glycosidtransferase and starch in the presence of Bacillus.

Various studies using acute, sub-acute and chronic toxic tests have shown that the macromolecular substance is non-toxic. Subsequent to the micro-encapsulation process, each granule may be coated with at least one and preferably a plurality of layers of the coating materials described above. The following provides a more detailed description of a method of preparing the cysteamine-containing composition used in the present invention.

In a jacketed reactor linked with polytetrafluoroethylene and equipped with a polytetrafluoroethylene coated stirrer, 4080 g of 75 wt % cysteamine hydrochloride solution in ethanol is added with mainly nitrogen being the atmosphere. The purity, melting point and burning residue of the cysteamine used are preferably 98% or above, 66 to 70° C. and 0.05% or below respectively. 1200 g β-cyclodextrin is then added into the reactor similarly under the protection of nitrogen gas. (The quality of β-cyclodextrin is in accordance with the requirements for a food additive. In particular, the dry basis purity is more than 98%; the weight loss by drying is less than 10.0%; the burning residue is less than 0.2%; the content of heavy metal is less than loppm; the arsenic content is less than 2 ppm.) The mixture is then heated for 3 hours at 40° C. Heating is then stopped and stirring continues for two hours thereafter, products resulted therefrom are then grounded and sieved through a screen (e.g. 40-mesh) filter after the products have been vacuum dried at a temperature of 40-50° C. All parts of the equipment, which may come in contact with the ingredients of the composition, should preferably be made of stainless steel.

In a tank-type mixer, 4200 g (on dry basis) of the cysteamine which has undergone the inclusion process as described, 2600 g of the fillers, and 1200 g of the disintegrants and 1700 g binders are added under the protection of a dry surroundings. These ingredients are then thoroughly mixed, and a suitable amount of anhydrous ethanol may be added and then mixed therewith. The resulting mixture presents a soft material with moderate hardness, so that it can be shaped into a ball by a light hold of palms. The ball-shaped resulting mixture may then be broken up by a light touch. After the mixture is pelleted by a granulator under the protection of nitrogen, the small granules resulting therefrom are immediately introduced to a fluid-bed dryer, and are then dried at the temperature of 40-50° C. in a substantially vacuum environment. Enteric coating materials are then prepared by a method with the following formulation: cellulose acetate phthalate 8.0 g, polyethylene glycol terephthalate 2.4 ml, ethyl acetate 33.0 ml and isopropyl acetate 33.6 ml. The resultant granules obtained above are uniformly coated under the protection of nitrogen with at least one layer but preferably a plurality of layers of the enteric coating materials described above. The enteric coating materials are dissolvable only at an alkaline environment. This can prevent the cysteamine from prematurely escaping from the composition or otherwise being degraded while it is still in the stomach of the animal. Cysteamine can adversely stimulate gastric mucous of the stomach of the animals. It is however worthwhile mentioning that cysteamine has relatively little side effects otherwise.

The resultant granules of the cysteamine-containing composition are then dried completely in a vacuum dryer at a temperature of 40 to 50° C. Then, all solvents are removed. The resultant granules are then allowed to cool to room temperature, and the micro-capsula were mixed with a suitable amount of flavoring and smelling agents by a cantilever double helix blender. The cysteamine-containing composition is a microcapsule with its interior having cysteamine hydrochloride and cyclodextrin, and with its exterior coated with the enteric coating materials.

The composition produced will exhibit small granular (or micro-particulate) shape having smooth surface, good flow property, and is easy to blend with various animal feeds. The diameter of each granule of the composition is preferably 0.28 to 0.90 mm. The composition also has excellent stability. It has been found that after the composition is packaged with sealed plastic bags and stored for one year in a cool, dark and dry place, their properties remain unchanged. Therefore, they meet the requirements for a feed additive.

The composition having the particular construction described above has a number of functional advantages over cysteamine by itself. Firstly, the activity of the cysteamine contained in the composition is preserved after it has been produced. This is important as the composition may be used as a feed additive and stored for a relatively long period of time before use. Secondly, the composition does not cause any noticeable gastro side effects to the animals fed therewith. Thirdly, the activity of the composition is preserved not only during storage but more importantly until it reaches the intestines of the animals. Fourthly, the composition can be easily administered to farm animals on a large-scale basis cost-effectively because it can be readily mixed with any basal feed. No separate procedure or injection is needed at all.

Various experiments have been conducted to demonstrate that administering a feed having cysteamine or a cysteamine-containing composition improves immunity of animals, two experiments of which are described in detail as follows.

### EXPERIMENTS

Experiment 1

Background Information

One hundred Holstein cows were used in the experiment. The average weight of the cows was about 600 kg. The cows were pre-treated with two days of the cysteamine-containing composition as described above. The actual experiment lasted for thirteen weeks.

Materials

The cysteamine-containing composition being in mini-pill form comprised about 30 wt % cysteamine together with other ingredients including cyclodextrin which served as a stabilizer. The content of cyclodextrin in the composition was 10 wt %. The composition was prepared by Walcom Bio-Chemicals Industry Limited.

Procedure

The cows were divided into a test group and a control group. The test group was administered with a predetermined amount of the cysteamine-containing composition via their cornmeal diet and the control group was administered with no cysteamine-containing composition. The cows were fed three times daily at 0730, 1430 and 2130.

Blood samples were collected from the cows at the end of the fifth-week treatment period by caudal vein puncture. 2 ml of each of the blood samples were centrifuged at 1500 rpm for 15 minutes. The supernatants were pooled and stored at −20° C. for IL-2 analysis. IL-2 concentration was determined with IL-2 RIA Kit (produced by Institute of Radioimmunological Technique in Military Hospital, China). The blood samples of the cows were also taken to determine the number of leukocytes. IL-6 concentration was similarly determined.

Data from the test and control groups of cows were analyzed and presented as MEAN +STANDARD ERROR. Results from the two groups of cows were compared by T-test.

Results and Discussion

The test group of cows administered with 30 g/day of the cysteamine-containing composition had about 29% higher serum concentration of IL-2 than that of the control group of cows. Table 1 below summarizes the levels of IL-2, IL-6 and leukocytes of the two groups of cows.

TABLE 1

| | IL-2 | Leukocytes | IL-6 in |
|---|---|---|---|
| | (ng/ml) | 10⁹/L | plasma (pg/ml) |
| Test cows | 5.5 ± 0.29 | 9.12 | 309.36 ± 20.98 |
| | (n = 50) | (n = 20) | (n = 37) |
| Control | 4.3 | 10.8 | 251.86 ± 20.98 |
| cows | (n = 50) | (n = 15) | (n = 39) |

The 29% increase in IL-2 concentration is remarkably significant statistically (P<0.01). It is to be noted that the number of peripheral blood leukocytes was also determined but it was found that there was no significant difference thereof between the test and control groups of cows. This indicates that the high concentration of IL-2 detected was stimulated by the cysteamine-containing composition and not by other factors.

The results also show that the test group of cows had a 22.8% higher plasma concentration of IL-6.

The experimental results indicate that cysteamine modulates and strengthens the immune system of animals by increasing the levels of IL-2 and IL-6.

Experiment 2

Background Information

Sixty-four Holstein cows were used in the experiment. The cows were randomly divided into a test group and a control group, having thirty-two cows in each group.

Materials

The same cysteamine-containing composition used in Experiment I was used in this experiment.

Procedure

Starting from about twenty weeks after calving, each cow in the test group was administered with an initial amount of 20 g/day of the cysteamine-containing composition. The dosage of the cysteamine-containing composition was then gradually increased stepwise until 60 g/day and then decreased to 30 g/day towards the end of the experiment. The treatment with the cysteamine-containing composition lasted for 140 days. The cows were administrated with the cysteamine-containing composition via its diet. The diet comprises cornmeal powder. The control group of cows was not administered with any cysteamine-containing composition. The cows were fed three times daily.

At the end of the 140-day experimental period, blood samples of the two groups of cows were obtained through their caudal veins. The blood samples were mixed with heparin for anticoagulation. The blood samples were incubated in a culture medium (RMPI 1640) and the white blood cells were isolated therefrom.

The isolated white blood cells from the samples of from the test group of cows were divided into two batches, with one batch added with phytohemagglutnin (PHA) and the other batch added without any PHA. The two batches of blood samples were then added with ³H-TdR and incubated for seventy-two hours.

The samples were then measured for the level of ³H-TdR incorporation by scintillation counter. The level of ³H-TdR incorporation was expressed in cpm.

The stimulation Index (SI) was then calculated based on the following formula

$$SI = \frac{mean cpm in T \text{-} cell cultured added with PHA}{mean cpm in T \text{-} cell cultured without PHA}$$

The blood samples from the control group of cows were similarly treated and the SI thereof was calculated.

Results and Discussion

Table 2 below summarizes the average SI values of the two groups of cows.

TABLE 2

Group

| Index | Test | Control |
|---|---|---|
| Stimulation Index (SI) | 29.73 ± 3.53* | 21.85 ± 2.07 |

*p < 0.05

As shown in the Table 2, the SI index of test group of cows was 29.73±3.53 which is 36% higher than test of the control group of cows. The higher SI index means that there is a higher number of lymphocytes in the blood of the test group of cows indicating a generally improved immunity.

From the above two experiments, it is shown that cysteamine, or the cysteamine-containing composition, has effect in improving immunity of animals. In particular, cysteamine, or the cysteamine-containing composition has effects in increasing the level of interleukin-2 and also stimulating production of T-cells.

Further studies have shown that cysteamine is particularly effective in vertebrate animals in improving their immunity.

It is envisaged that with an improved immunity, farm with farm animals will be more productive. For example, farms for raising meat-producing cattle will have a higher yield due to a lower death rate of the animals because of the improved immunity thereof. It is also envisaged that cysteamine or a cysteamine-containing composition according to the present invention may also be used in humans for improving immunity of patients. For example, cysteamine may be used in combination with a conventional AIDS pharmaceutical for treating HIV infected patients.

The contents of each of the references discussed above, including the references cited therein and the references listed below, are herein incorporated herein in their entirety. It is to be noted that numerous variations, modifications, and further embodiments are possible and accordingly, all such variations, modifications and embodiments are to be regarded as being within the scope of the present invention.

### REFERENCES

1. Besedovsky H O, del Rey A. 1992.Immune-neuroendocrine circuits: integrative role of cytokines. Front Neuroendocrinol 13(1): 61-94
2. Auernhammer C J, Strasburger C J. 1995. Effects of growth hormone and insulin-like growth factor I on the immune system. Eur J Endocrinol 133(6): 635-45
3. Khosraviani M, Davis S L. 1996. Hormonal regulation of peripheral blood mononuclear cells in sheep. Domest Anim Endocrinol 13(2): 139-50
4. Torre P M, Lewis M J, Ingle T L, Oliver S P. 1993. Influence of recombinant bovine somatotropin (sometribove) on mononuclear cells during the nonlactating period. J Dairy Sci 1993 Apr; 76(4): 983-91
5. Szabo S, Reichlin S. 1981. Somatostatin in rat tissues is depleted by cysteamine administration. Endocrinology 109(6): 2255-7
6. Millard W J, Sagar S M, Badger T M, Martin J B.1983. Cysteamine effects on growth hormone secretion in the male rat. Endocrinology 112(2): 509-17
7. Mcguffet R K, Green H B, Basson R P, Ferguson T H. 1990. Lactation response of dairy cows receiving bovine somatotropin via daily injection or in a sustained-release vehicle. J Dairy Sci. 73: 763-771

1-52. (canceled)

53. The use of cysteamine, a salt thereof, or a composition containing cysteamine or a salt thereof, for improving immunity of animals.

54. The use according to claim 53, wherein said improving immunity includes increasing the level of interleukin-2 (IL-2) in said animals.

55. The use according to claim 53, wherein said improving immunity includes increasing the level of interleukin-6 (IL-6) in said animals.

56. The use according to claim 53, wherein said improving immunity includes stimulating production of lymphocytes in said animals.

57. The use according to claim 53, wherein said animals are vertebrate animals.

58. The use according to claim 53, wherein said composition comprises 1 to 95 wt % of cysteamine, or a salt thereof, and an inclusion compound host materials composition comprising a stabilizer selected from a group including cyclodextrin and its derivatives.

59. The use according to claim 58, wherein said cysteamine-containing composition comprises 1 to 60 wt % of said inclusion compound host materials composition.

60. The use according to claim 53, wherein said cysteamine-containing composition comprises at least one of fillers, disintegrants, binders, and flavorings and smelling agents.

61. The use according to claim 53, wherein said cysteamine-containing composition comprises coating materials.

62. The use according to claim 61, wherein said cysteamine-containing composition comprises 1 to 20 wt % of said coating materials.

63. The use according to claim 61, wherein said coating materials are enteric.

64. The use according to claim 61, wherein said coating materials are selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymer and formogelatine.

65. The use according to claim 60, wherein said cysteamine-containing composition comprises 1 to 90 wt % of said fillers.

66. The use according to claim 60, wherein said fillers are selected from a group including powdered cellulose, starch and calcium sulfate.

67. The use according to claim 60, wherein said cysteamine-containing composition comprising 5 to 50 wt % of said binders and said disintegrants.

68. The use according to claim 60, wherein said binders and said disintegrants are selected from a group including hydropropyl starch, microbial alginate, microcrystalline cellulose and starch.

69. The use according to claim 60, wherein said cysteamine-containing composition comprises 0.05 to 0.3 wt % of said flavoring and smelling agents for enhancing the flavor thereof.

70. The use according to claim 61, wherein said cysteamine-containing composition is formed into granules, each of which comprises at least one layer of said coating materials.

71. The use according to claim 58, wherein said cysteamine-containing composition is formed into granules in which the cysteamine or a salt thereof is shielded from its surroundings by said inclusion compound host materials composition.

72. The use according to claim 70, wherein each of said granules of said cysteamine-containing composition has a size ranging from 0.28 to 0.90 mm in diameter.

73. The use according to claim 63, wherein said cysteamine-containing composition is encapsulated by said enteric coating materials.

74. The use according to claim 53 for the manufacture of an animal feed additive.

75. The use according to claim 53 for the manufacture of an animal feed.

76. An immunity improving composition for animals, comprising cysteamine or a salt thereof.

77. A composition according to claim 76, wherein said improving immunity includes increasing the level of interleukin-2 (IL-2) in said animals.

78. A composition according to claim 76, wherein said improving immunity includes increasing the level of interleukin-6 (IL-6) in said animals.

79. A composition according to claim 76, wherein said improving immunity includes stimulating production of lymphocytes in said animals.

80. A composition according to claim 76, wherein said animals are vertebrate animals.

81. A composition according to claim 76, comprising 1 to 95 wt % of cysteamine, or a salt thereof, and an inclusion compound host materials composition comprising a stabilizer selected from a group including cyclodextrin and its derivatives.

82. A composition according to claim 81, wherein said cysteamine-containing composition comprises 1 to 60 wt % of said inclusion compound host materials composition.

83. A composition according to claim 76, wherein said cysteamine-containing composition comprises at least one of fillers, disintegrants, binders, and flavorings and smelling agents.

84. A composition according to claim 76, wherein said cysteamine-containing composition comprises coating materials.

85. A composition according to claim 84, wherein said cysteamine-containing composition comprises 1 to 20 wt % of said coating materials.

86. A composition according to claim 84, wherein said coating materials are enteric.

87. A composition according to claim 84, wherein said coating materials are selected from a group including cellulose acetate phthalate, starch acetate phthalate, methyl cellulose phthalate, glucose or fructose derivatives from phthalic acid, acrylic and methacrylic copolymers, polymethyl vinyl ether, partly esterified substance of maleic anhydride copolymer and formogelatine.

88. A composition according to claim 83, wherein said cysteamine-containing composition comprises 1 to 90 wt % of said fillers.

89. A composition according to claim 83, wherein said fillers are selected from a group including powdered cellulose, starch and calcium sulfate.

90. A composition according to claim 83, wherein said cysteamine-containing composition comprising 5 to 50 wt % of said binders and said disintegrants.

91. A composition according to claim 83, wherein said binders and said disintegrants are selected from a group including hydropropyl starch, microbial alginate, microcrystalline cellulose and starch.

92. A composition according to claim 83, wherein said cysteamine-containing composition comprises 0.05 to 0.3 wt % of said flavoring and smelling agents for enhancing the flavor thereof.

93. A composition according to claim 84, wherein said cysteamine-containing composition is formed into granules, each of which comprises at least one layer of said coating materials.

94. A composition according to claim 81, wherein said cysteamine-containing composition is formed into granules in which the cysteamine or a salt thereof is shielded from its surroundings by said inclusion compound host materials composition.

95. A composition according to claim 93, wherein each of said granules of said cysteamine-containing composition has a size ranging from 0.28 to 0.90 mm in diameter.

96. A composition according to claim 86, wherein said cysteamine-containing composition is encapsulated by said enteric coating materials.

97. An immunity improving animal feed additive for improving immunity of animals comprising cysteamine, a salt thereof, or a cysteamine-containing composition as defined in claim 76.

98. An immunity improving animal feed for improving immunity of animals comprising a cysteamine, a salt thereof, or a cysteamine-containing composition as defined in claim 76.

99. A method of improving immunity of an animal, the method comprising administering a composition comprising cysteamine or a salt thereof to the animal.

100. The method according to claim 99, wherein improving immunity includes increasing the level of interleukin-2 (IL-2) in the animal.

101. The method according to claim 99, wherein improving immunity includes increasing the level of interleukin-6 (IL-6) in the animal.

102. The method according to claim 99, wherein improving immunity includes stimulating production of lymphocytes in the animal.

103. The method in according to claim 99, wherein the composition comprises 1 to 95 wt % of cysteamine, or a salt thereof, and inclusion compound host materials comprising a stabilizer selected from a group including cyclodextrin and its derivatives

104. The method according to claim 103, wherein said composition comprises 1 to 60 wt % of said inclusion compound host materials.

105. The method according to claim 99, wherein said composition comprises at least one of fillers, disintegrants, binders and flavorings and smelling agents.

106. The method according to claim 99, wherein the composition comprises coating material.

107. The method according to claim 106, wherein the composition comprises 1 to 20 wt % of said coating material.

108. The method according to claim 106, wherein said coating material is enteric.

109. The method according to claim 106, wherein said coating material is selected from a group including cellulose acetate phthalate, starch acetate phthalate, methyl cellulose phthalate, glues or fructose derivatives from phthalic acid, acrylic and methacrylic copolymers, polymethl vinyl ether, partly esterified substance of maleic anhydride copolymer and formogelatine.

110. The method according to claim 105, wherein said composition comprises 1 to 90 wt % of said fillers.

111. The method according to claim 105, wherein said fillers are selected from a group containing powdered cellulose, starch and calcium sulfate.

112. The method according to claim 105, wherein said composition comprises 5 to 50 wt % of said binders and said disintegrants.

113. The method according to claim 105, wherein said binders and said disintegrants are selected from a group including hydropropyl starch, microbial alginate, microcrystalline cellulose and starch.

114. The method according to claim 105, wherein said composition comprises 0.05 to 0.3 wt % of said flavoring and smelling agents for enhancing the flavor thereof.

115. The method according to claim 106, wherein said composition is formed into granules, each of which comprises at least one layer of said coating material.

116. The method according to claim 106, wherein said composition is formed into granules in which the cysteamine or a salt thereof is shielded from its surroundings by said inclusion compound host material.

117. The method according to claim 115, wherein each of said granules of said composition has a size ranging from 0.28 to 0.90 mm in diameter.

118. The method according to claim 108, wherein said composition is encapsulated by said coating material.

119. The method according to claim 99, wherein the animal is a vertebrate.
